(19) United States
(12) Patent Application Publication
(10) Pub. No.: US 2005/0106720 A1
(43) Pub. Date: May 19, 2005

(54) Device and method for cultivating tissue cells

(76) Inventors: Stephanie Nagel-Heuer, Hamburg (DE); Ralf Portner, Buxtehude (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/505,896

(22) PCT Filed: Feb. 27, 2003
(86) PCT No.: PCT/DE03/00668

(30) Foreign Application Priority Data
Feb. 27, 2002 (DE) 102 08 311.8

(51) Int. Cl. C12N 5/06; C12N 5/08

(52) U.S. Cl. 435/325; 435/366

(57) ABSTRACT

The invention relates to a device and a method for cultivating tissue cells. According to the invention, the tissue cells are cultivated in a culture zone, a thin layer of nutrient solution flowing across the tissue cells. The inventive method is particularly useful for propagating implantable cells such as skin or bone tissue cells and cartilage or vessel cells. The inventive method is further suitable for obtaining implantable cartilage or bone constructs.

The invention relates to a process and a device for cultivation of tissue cells.

The cultivation of tissue cells plays a role in the so-called “Tissue Engineering.” In this case, it is the purpose to create artificial cell tissue with body-specific properties. In many cases, cells are cultivated on certain biomatrices (structurates). Applications for “Tissue Engineering” are, e.g., implant production (generation of artificial skin, functional vessels or tissue systems (liver, cartilage, etc.)), physiological studies of “in vitro” tissue cultures (medium, metabolism, etc.), compatibility studies of biomaterials, compatibility tests of medications or toxicity tests for certain substances.

In the production of functioning tissues, the procedure can be performed in several steps, whereby important points are the control of the differentiation in cultivated tissue and a specific geometric structure of the implant (e.g., skin—large-area, cartilage replacement for ear trauma—three-dimensional structure, etc.). In a first step, the cells that are removed in a biopsy are reproduced in bottle cultures in a special nutrient medium to increase the number of cells.

For advanced cultivation, a possible concept calls for applying the cells on a special tissue base. For this purpose, these can be filter bases, fleeces or matrices with a sponge structure that optionally consist of biodegradable polymers. The thus created tissues are then cultivated until a tissue with the desired properties has formed.

In principle, two cultivation methods can be distinguished. The most commonly used method is the cultivation under so-called static conditions in special culture bottles (T-bottle, 12-well plate, etc.), which are placed in a special incubator with appropriate temperature equalization and an atmosphere that is concentrated with carbon dioxide. In this case, the consumed nutrient medium is exchanged at specific intervals for fresh nutrient medium. Gasification (supply with oxygen) is usually carried out from the atmosphere of the gasifying cabinet. Drawbacks of these cultivation methods are the stationary conditions relative to the media components as well as the very large amount of manual labor, which involves a high risk of contamination.

As an alternative, the tissues can be introduced into a bioreactor (a so-called perfusion chamber), through which culture medium flows continuously and in which an improved and controlled supply with substrates and oxygen as well as a removal of metabolic products can take place. In this case, the culture medium can be pumped out from a gasified receiving vessel into a circuit or alternatively can be discarded after passing once through the perfusion chamber.

DE-A1 198 08 055 describes an example of such a perfusion chamber. In the apparatus described there, however, there exists the drawback that the chamber must be filled completely with liquid in order to operate as designed. In this connection, the danger exists that gas bubbles that are contained in the liquid collect in the chamber and prevent the flow through the chamber. In addition, in perfusion chambers, the spatial arrangement of the tissue media that are used ensures the quick removal of oxygen over the length of the chamber, by which the danger occurs that the rear tissue cells can no longer be adequately supplied with gas, in particular with oxygen.

The object of this invention is therefore to provide a process and a device for cultivation of tissue cells with which the described drawbacks can be eliminated. In this case, the tissue cells are to be able to be supplied adequately with gas and nutrient medium.

According to the invention, the achievement of the set object is carried out according to the characterizing portions of claims 1 and 16.

The invention offers the advantage that an optimal supply of tissue cells both with nutrients and with gaseous substances is made possible by the flow layer that is formed above the tissue cells. In this way, fresh medium can get into the tissue cells. Moreover, the gas supply to the tissue cells is improved, since the diffusion paths for the gases are small.

According to a preferred embodiment of the invention, a gas stream is produced, which is oriented in the direction opposite the direction of flow of the nutrient. In this connection, it is ensured primarily in an arrangement of several tissue cultures that all tissue cultures are adequately supplied with gas, in particular with oxygen, and that it does not result in any undesired removal of oxygen over the length of the culture area.

Moreover, the diffusion path for the gases can be set by the layer thickness of the nutrient, for example by the formation of an overflow edge that is described in the embodiments.

According to a preferred embodiment of the invention, the thin layer of the nutrient medium above the tissue cells is 0.1 . . . 3.0 mm, preferably 0.5 . . . 1.0 mm.

The formation of a thin nutrient medium layer above the tissue cells can preferably be achieved in that nutrient medium is sent into a culture area in which the tissue cells are found. With the nutrient medium, an overflow from the culture area is then created, and the nutrient medium goes into a collecting chamber after flowing over the tissue cells. The nutrient medium is then drawn off again from the collecting chamber.

Other embodiments of the invention are described in the subclaims.

The process according to the invention is suitable in particular for cultivation of human, animal and plant cells. Depending on the type of cells used, one skilled in the art knows which nutrient medium is necessary for cultivation. The nutrient medium can be made up accordingly. The same applies for the use of necessary gases. If, i.a., oxygen is required in human and animal cells, generally a need for carbon dioxide arises in plant cells. Depending on the type of gases used, it may also be appropriate to adapt the composition of the nutrient medium thereto. Thus, for example, the need for an elevated buffer capacity may arise or a pH regulation may be necessary.

Moreover, the process according to the invention is suitable for reproducing implantable cells. Cells that are implanted in human or animal bodies are in particular skin or bone tissue cells as well as cartilage and vessel cells.

Moreover, the process is suitable for obtaining implantable cartilage constructs or bone constructs. Specifically for obtaining such constructs, the process according to the invention offers the advantage that the tissue cells occupy three-dimensional structures but still can be supplied adequately with nutrient medium and oxygen.

Also, the process according to the invention is ultimately suitable for performing tests of effect and toxicity. In this way, the action of medications, environmental toxins and the like on tissue cells can be studied to make possible, in so doing, an alternative to animal tests. In this case, according to its respective aggregate state, the substance that is to be studied can either be used in the gas phase or added to the nutrient medium in solid or liquid form.

Preferred embodiments of the invention are described in more detail below based on the drawings.

Here:

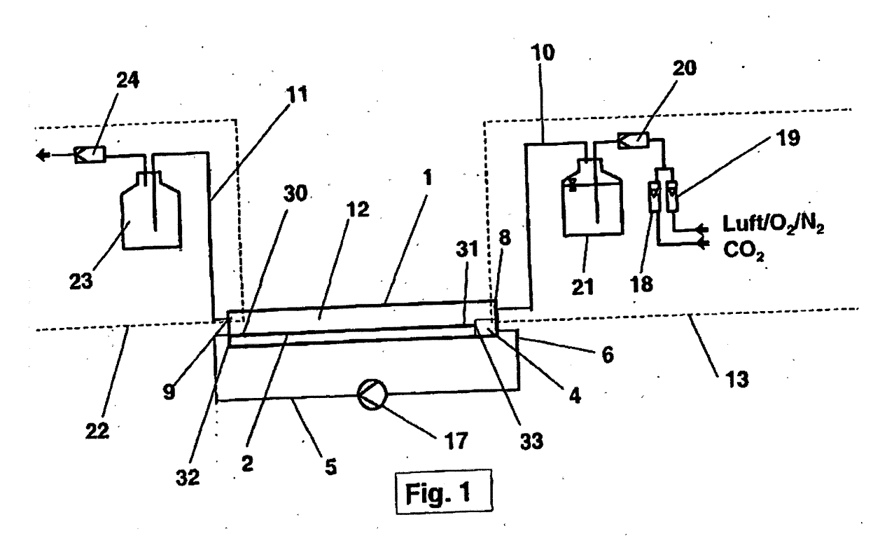

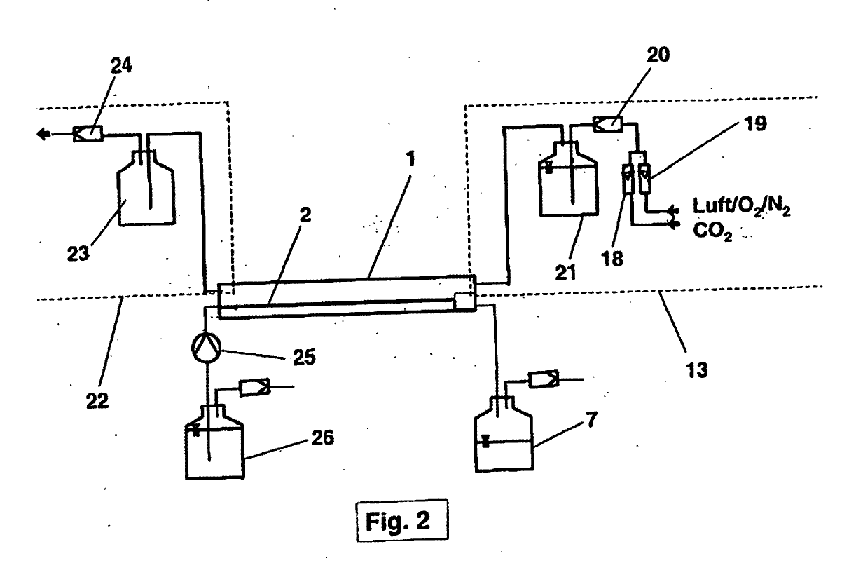

FIGS. 1 and 2 show a diagrammatic visualization of a treatment apparatus with a gas supply unit and an exhaust air line connected thereto,

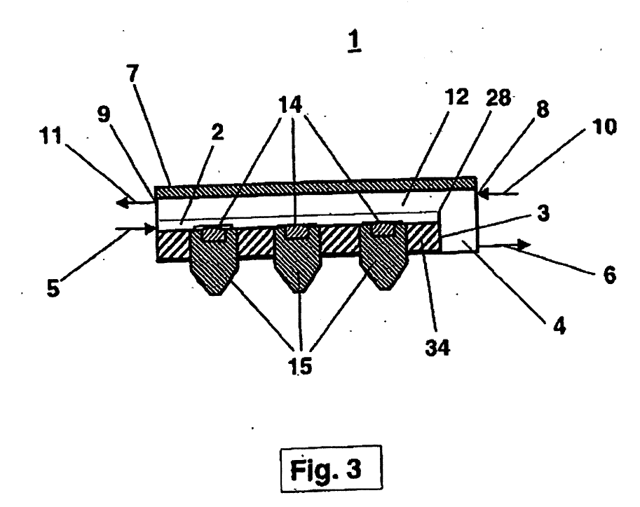

FIG. 3 shows a diagrammatic visualization of a treatment apparatus with individual inserts,

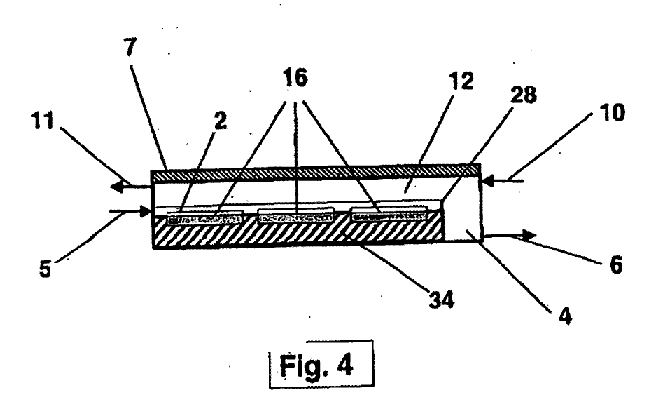

FIG. 4 shows a diagrammatic visualization of a treatment apparatus with media for adherent cell cultures,

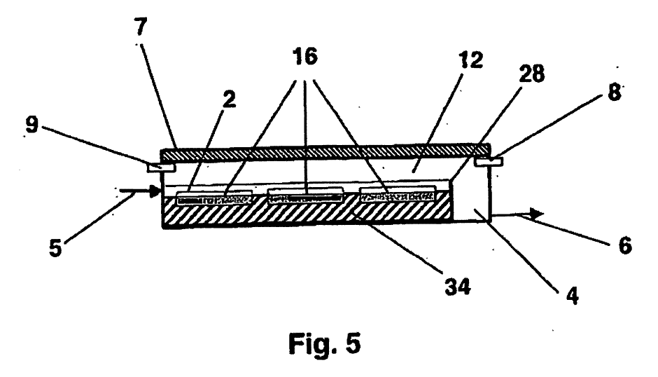

FIG. 5 shows a diagrammatic visualization of a treatment apparatus without a special gas line, and

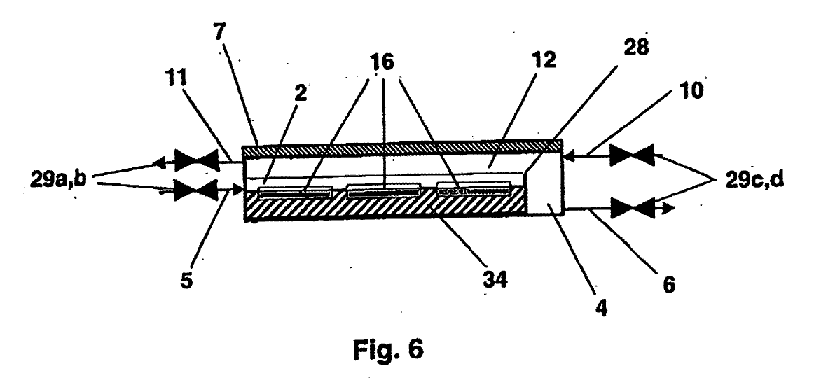

FIG. 6 shows a diagrammatic visualization of a treatment apparatus for pressurization.

In FIG. 1, a device for cultivating tissue cells with a treatment apparatus I is depicted, in which treatment apparatus 1 has a culture area 2 in which tissue cells that are not depicted in more detail are brought into contact with a nutrient medium. In this connection, treatment apparatus 1 has an inlet 32 and an outlet 33 for the nutrient medium, such that the nutrient medium can flow from one end 30 of culture area 2 to other end 31. Then, the nutrient medium passes into a collecting chamber 4. From collecting chamber 4, the nutrient medium is drawn off via line 6. A pump 17 transports the nutrient medium in the circuit via line 5 back into culture area 2. With the aid of pump 17, the flow rate of the nutrient medium can be regulated such that in particular the flow rate of the nutrient medium above the tissue cells can be set. In addition, FIG. 1 calls for a gas supply unit 13, with the aid of which a definable mixture of various gases can be produced from, for example, air, oxygen, nitrogen and carbon dioxide and can be supplied to treatment apparatus 1. Gas supply unit 13 can also have flowmeters 18, 19 as well as a sterile filter 20. Wetting agents 21 can also be provided to humidify the gas with water before introduction into treatment apparatus 1. Via line 10, the gas moves through gas intake opening 8 into interior space 12 of treatment apparatus 1. In this connection, the nutrient medium that is contained in culture area 2 is supplied with gas. The gas here flows counterclockwise to the flow of the nutrient medium over the nutrient medium and leaves interior space 12 of treatment apparatus 1 through gas exhaust opening 9. A line 11 is connected to gas exhaust opening 9 via which the gas is conveyed to an exhaust air line 22. The exhaust air line contains a sterile trap 23 as well as an exhaust air filter 24.

Since it turned out that the growth of the cells can be influenced by stimulation of the shear stress, the flow rate of the nutrient also has an influence on the growth of the tissue cells. In the test setup selected in FIG. 1, up to 5 ml of nutrient medium was conveyed per minute. The output was preferably 0.25 to 1 ml/minute. In the test setup depicted in FIG. 2, only up to 30 ml/day, preferably 2.5 to 10 ml/day, was conveyed.

According to the embodiment of the invention depicted in FIG. 2, fresh nutrient medium from a storage bottle 26 is constantly sucked in by means of a pump 25 and directed into culture area 2 of treatment apparatus 1. Consumed medium is collected in a receiver bottle 27.

FIG. 3 shows treatment apparatus 1 in magnified visualization. Supply and discharge pipes for gas and nutrient medium are characterized by arrows. Treatment apparatus 1 exhibits a bottom section 34, which is provided for receiving media 14, 16 for the tissue cells. An overflow edge 28 is created on bottom section 34 via which the nutrient medium can flow from culture area 2 into a collecting chamber 4. Overflow edge 28 is formed in the embodiment shown by an elevated side wall 3 of bottom section 34. In addition, this embodiment exhibits the special feature that special inserts 15 are provided for pre-structured three-dimensional media 14 that can be compact or macroporous. In this connection, inserts 15 are detachably connected with bottom section 34. They can be screwed in preferably from below into bottom section 34. In the installed state of inserts 15, the tissue cells are then positioned such that a thin layer of nutrient medium can flow over them. After flowing over the tissue cells, the nutrient then flows into collecting chamber 4.

Media 14 that are depicted in FIG. 3 are preferably arranged in one or two series in a flow canal that is not depicted in more detail. The width of the flow canal can be 5 to 7 cm. Under certain circumstances, larger widths have the drawback that no uniform flow profiles can be formed in the flow canal. However, the length of the flow canal in principle does not play any role. If possible, however, it should not be larger than 20 to 25 cm, such that about 5 to 10 media 14 can be placed in the flow canal.

As a further special feature, the embodiment that is depicted in FIG. 4 shows special media 16 that are designed for adherent cell cultures. Media 16 preferably consist of glass or suitable plastics. They are positioned according to FIG. 3 like media 14 such that the nutrient medium, in a thin layer, can flow through the tissue cells that are contained in inserts 16 and can move into collecting chamber 4.

In contrast to the embodiments according to FIGS. 1 to 4, FIG. 5 shows a treatment apparatus 1, in which gas moves into interior space 12 in the path of diffusion. To this end, as a gas intake opening 8, a slit-like opening is provided in upper portion 7 that can also be closed with a diaphragm to avoid contamination. The equivalent holds true for gas exhaust opening 9. In addition, supply and discharge pipes for the nutrient medium exist. They are characterized by arrows. For the embodiment according to FIG. 5, the advantage arises that a device with such a treatment apparatus 1 does not require any special gasifying agents. The cultivation of tissue cells can be performed in an incubator in this case without additional equipment.

In FIG. 6, ultimately one embodiment with a treatment apparatus 1 is depicted, in which interior space 12 is pressurized. A defined overpressure can be set via suitable valves 29 a-d by which, for example, the transition of gaseous substances into the nutrient medium is facilitated and thus the supply of tissue cells with these substances is improved.

### LEGEND

1 Treatment apparatus
2 Culture area
3 Side wall
4 Collecting chamber
5 Line
6 Line
7 Upper portion
8 Gas intake opening
9 Gas exhaust opening
10 Line
11 Line
12 Interior space
13 Gas supply unit
14 Medium
15 Insert
16 Medium
17 Pump
18 Flowmeter
19 Flowmeter
20 Sterile filter
21 Wetting agent
22 Exhaust air line
23 Sterile trap
24 Exhaust air filter
25 Pump
26 Storage bottle
27 Receiver bottle
28 Overflow edge
29 a-d valves
30 End
31 End
32 Feed
33 Discharge
34 Bottom section

1. Process for cultivating tissue cells, in which the tissue cells are supplied with nutrient medium and gas, characterized in that the tissue cells are cultivated in a culture area and coated with nutrient from one end of the culture area to the opposite end of the culture area so that a thin nutrient medium layer, which is supplied with gas, is formed above the tissue cells.

2. Process according to claim 1, wherein a gas stream is created that runs counterclockwise to the direction of flow of the nutrient medium.

3. Process according to claim 1, wherein the layer thickness of the nutrient medium above the tissue cells can be adjusted.

4. Process according to claim 3, wherein the layer thickness of the nutrient medium above the tissue cells is 0.1 . . . 3.0 mm, preferably 0.5 . . . 1.0 mm.

5. Process according to claim 1, wherein an overflow of nutrient medium is created on one end of the culture area such that the nutrient medium flows into a collecting chamber after flowing over the tissue cells.

6. Process according to claim 5, wherein nutrient medium is drawn off from the collecting chamber and returned to the culture area in the circuit.

7. Process according to claim 1, wherein air or another gas that is used to supply the tissue cells is used as a gas.

8. Process according to claim 1, wherein the treatment apparatus is pressurized.

9. Process according to claim 1 for cultivation of human, animal or plant cells.

10. Process according to claim 1 for reproducing implantable cells.

11. Process according to claim 1 for obtaining implantable cartilage constructs.

12. Process according to claim 1 for obtaining implantable bone constructs.

13. Process according to claim 1, in which an active ingredient, whose action on the tissue cells is to be examined, is added to the nutrient medium.

14. Process according to claim 1, in which the gas contains an active ingredient whose action on the tissue cells is to be examined.

15. Process according to claim 1 with the purpose of the production of substances that are formed by the tissue cells.

16. Device for cultivating tissue cells with a treatment apparatus in which the tissue cells are supplied with gas and nutrient medium, whereby the treatment apparatus has a feed and a discharge for the nutrient medium, wherein treatment apparatus (1) contains a culture area (2) with an arrangement of media (14, 16) for the tissue cells, such that the tissue cells can be positioned in such a way that the nutrient medium can flow into a thin layer above the tissue cells and that treatment apparatus (1) has an upper portion (7) that is provided with a gas intake opening (8) and a gas exhaust opening (9).

17. Device according to claim 16, wherein gas intake opening (8) and gas exhaust opening (9) are arranged such that in the case of a gas line in treatment apparatus (1), a gas stream that runs counterclockwise to the direction of flow of the nutrient medium is formed.

18. Device according to claim 17, wherein the layer thickness of the nutrient medium above the tissue cells is 0.1 . . . 3.0 mm, preferably 0.5 . . . 1.0 mm.

19. Device according to claim 16, wherein treatment apparatus (1) has a bottom section (34) for receiving media (14, 16) on which an overflow edge (28) is formed, via which the nutrient medium can flow into a collecting chamber (4).

20. Device according to claim 16, wherein a flow canal, in which media (14, 16) are arranged in series, is formed in bottom section (34).

21. Device according to claim 16, wherein lines (5, 6) are provided for the nutrient medium, whereby line (5) forms a feed line for the nutrient medium, and line (6) is connected to collecting chamber (4).

22. Device according to claim 16, wherein a line (10), which is connected to a gas supply unit (13), is connected to gas intake opening (8).

23. Device according to claim 16, wherein valves (29 a-d) for feeds and discharges of nutrient medium and gas are provided that make it possible to control pressure in interior space (12) of treatment apparatus (1).
